(19) United States
(12) Patent Application Publication
(10) Pub. No.: US 2008/0027290 A1
(43) Pub. Date: Jan. 31, 2008

(54) BASE STATION FOR RETRIEVING DATA FROM AND PROGRAMMING A MEDICAL DEVICE

(76) Inventor: TERRY KEITH BRYANT, SINGER ISLAND, FL (US)

Correspondence Address: DANIEL S. POLLEY, P.A., 1215 EAST BROWARD BOULEVARD, FORT LAUDERDALE, FL, 33301, US

(21) Appl. No.: 11/782,790

(22) Filed: Jul. 25, 2007

Related U.S. Application Data
(60) Provisional application No. 60/820,365, filed on Jul. 26, 2006.

(51) Int. Cl. A61B 5/00

(52) U.S. Cl. 600/300

(57) ABSTRACT

A base station is disclosed for serving as a translator between a medical device and a computer in order to retrieve data from and to program the medical device. The medical device is preferably already set up and programmed such that it is capable of receiving instructions through said base station. In one non-limiting embodiment the computer can be a personal computer and the medical device can be an Incentive Spirometer. The base station can preferably program the medical device to communicate in one of several available languages. Communication between the base station and the computer can be achieved through various wired and wireless technologies. In one non-limiting embodiment, communication is achieved through USB technology. In another non-limiting embodiment Bluetooth or WiFi wireless technologies can be used.

This application claims the benefit of and priority to U.S. Application Ser. No. 60/820,365, filed Jul. 26, 2006

### 1. FIELD OF THE INVENTION

The present invention relates generally to medical equipment and particularly to a novel way for retrieving data from and programming medical devices.

### 2. SUMMARY OF THE INVENTION

The present invention generally provides a base station serving as a translator between a medical device and a computer for retrieving data from and programming the medical devices. Though not considered limiting the computer can be a personal computer and the medical device can be an Incentive Spirometer. The base station can preferably program the medical device to communicate in one of several available languages. Communication between the base station and the computer can be achieved through various wired and wireless technologies. In one non-limiting embodiment, communication can be achieved through USB technology. However, any communication technology can be used and can be selected based on the requirements to performed or provide the desired functions of the base station, medical device and/or computer.

Preferably in order for the base station to appropriately transmit programmable instructions to any medical device, each medical device can be preferably assembled with technology such as through an electronic enhancement, which will be referred to for purposes of this application referred to as Medichip technology. This technology permits the medical apparatus to be in synthesis with the Base Station, thus permitting the programmer to appropriately exhibit whatever function of therapy required for the device to work through communicated language customized for each patient. These therapeutic exercises and functions of the medical device, as required, can preferably be emitted directly from said medical apparatus, and can include, without limitation: audible, verbal, commands, responses and prompting to the patient, as programmed by the Base Station, in order to help promote patient compliance when using said medical device. This compliance helps the patient follow those therapeutic procedures that each particular medical device advocates per the field of medicine the apparatus is being used for and through the Base Station, said apparatuses can be programmed according to the patience particular needs.

The use of the Medichip technology can provide the ability to construct each medical apparatus with a method of performing Verbal, commands, responses, instructions, and prompting, within and emanating from said medical apparatus in order to provide and advocate usage by the patient under the normal requirements for satisfactory compliance by the patient of each particular medical apparatus itself. Though the Medichip technology can be provided to allow the base station and the medial apparatuses to communicate unidirectional with each other for both programming the medial therapeutic performance and to retrieve data, such technology is not considered limited to only these purposes or functions. Accordingly, the Medichip technology can be used for providing any function which provides a way to produce compliance by the patient of the medical apparatus, and can be used in connection with the base station's function. As non-limiting examples, beeps, sounds, or noises, are also able to entice the patient to fulfill the therapeutic requirements and can also be programmed in accordance with the teachings of the present invention. Thus, any function similar to the above and below functions, is consider and encompassed as part of the function of the Medichip technology, in relationship to the base station and those applicable devices that have the ability to be programmed as medical apparatuses and all are also considered within the scope of the invention.

Though, the present invention is often described with the medical apparatus or medical device being an Incentive Spirometer, it should be recognized that the Incentive Spirometer is for example purposes and considered only one non-limiting embodiment for the medical apparatus or medical device. However, any medical apparatus or medical device incorporating technology which will allow it to be programmed through the base station is also considered within the scope of the invention. Additionally, the base station station is also considered within the scope of the invention. Additionally, the base station can be provided with a cradle-like member or coupling housing for receiving the programmable device. This cradle or coupling member can be configured or can be adjustable as is necessary to hold the particular one or more medical apparatuses that will be programmed or received data from through the base station.

### 2. BRIEF DESCRIPTION OF THE DRAWINGS

FIG. 1 illustrates a block diagram showing the base station in communication with a medical apparatus/medical device and personal computer in accordance with the present invention;

FIG. 2 illustrates a screen shot on the personal computer for communicating with the medical apparatus/medical device through the present invention base station;

FIG. 3 illustrates an alternative screen shot on the computer for communicating with the medical apparatus/medical device through the present invention base station;

FIG. 4 illustrates an alternative base station embodiment incorporating a USB port in accordance with the present invention;

FIG. 5 illustrates an interface software window in accordance with the present invention;

FIG. 6 illustrates one circuit board embodiment for the base station illustrating the pins which line up with the medical apparatus (i.e. incentive spirometer, etc.) pads;

FIG. 7 illustrates on circuit board embodiment for the medical apparatus (i.e. incentive spirometer, etc.) illustrating the pads which line up with the base station pins.

### 3. DESCRIPTION OF THE INVENTION

The present invention provides a base station for retrieving data from and programming a medical apparatus or medical device. Numerous types of medical apparatuses can benefit from and be used (such as, but not limited to, apparatuses having audible and/or verbal capabilities, etc.) with a novel base station of the present invention. The present invention base station communicates back and forth with the electronic supplementary assembly within the medical apparatus or medical device to allow or permit the two units to coincide for programming and providing data through a computer or similar unit. In accordance with the present invention, the base station can both receive and give information (data).

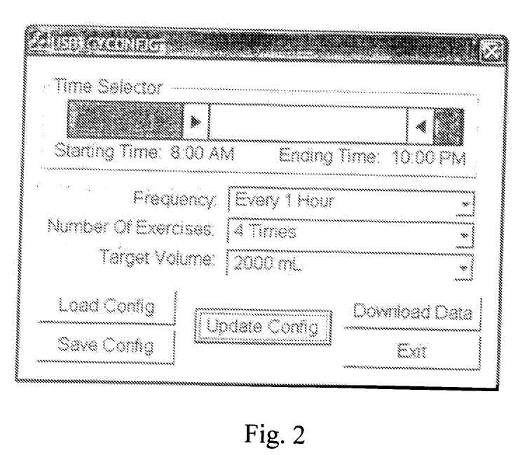

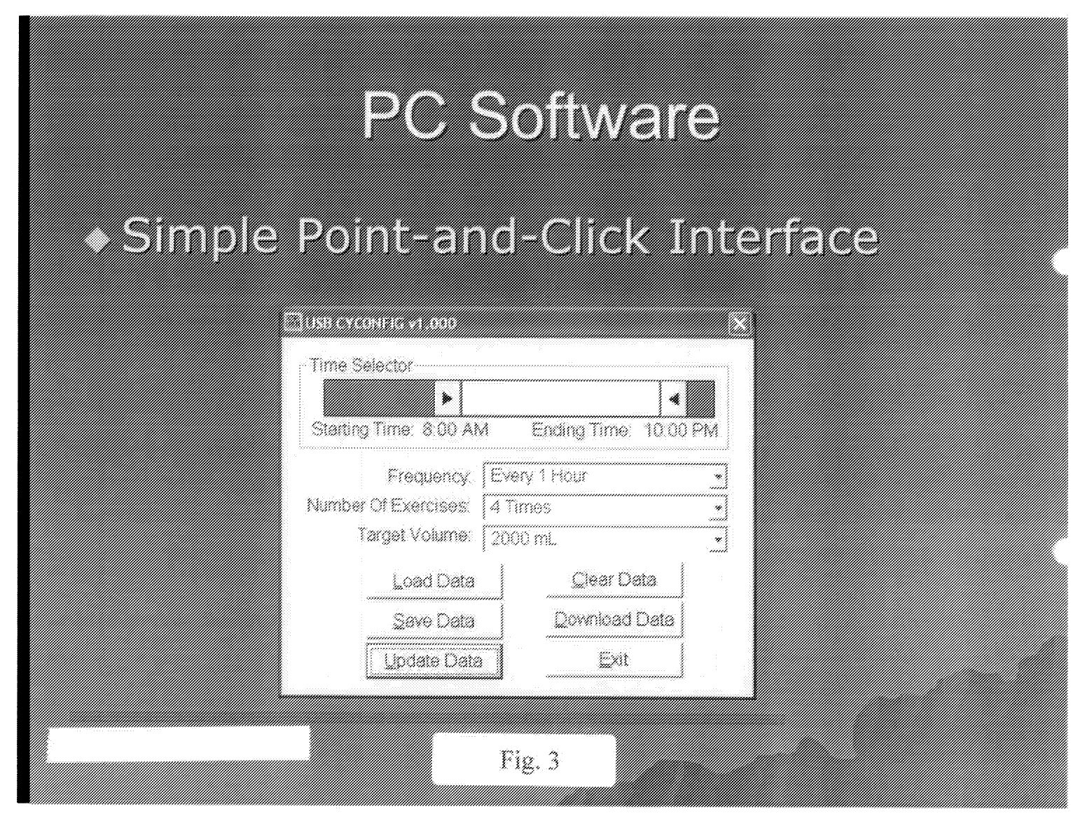

The base station does not require a “hard electronic” system to retrieve dates, other than the base itself, to put each of the medical devices in, which allows programming to be done. The only requirement is that the user has a computer, as all programming is done, (or information received), as seen on the computer screen, using the curser of the computer and this allows an output signal to go thru the computer to the base station. The computer is not limited to any particular type of computer (e.g. desktop, PDA, Palm Pilot, BLACKBERRY, cellphone, notebook, laptop, or other similar devices having a viewing screen all which are collectively referred through the applications as “computer” or “PC”). Thus the base station allows data to be retrieved and also programmed. The base station can communicate and be programmed in any language. The base station is not limited to any particular language and its programmable ability can be in various different languages. In one embodiment, the base station can be attached to any computer with a simple cord (as such) to produce a readable visual picture of the different programmable abilities, such as, but not limited to, how long between sessions (See FIG. 2 or FIG. 3). A mouse, keyboard or other controller can be to move the cursor on the computer screen in order to set the volumes, settings, measurements, and session or whatever. No large mechanical equipment needed. Data from the medical apparatus can be retrieved in a similar fashion.

As seen in FIG. 2 or FIG. 3, a screen shot for one configuration or embodiment of a PC or computer software interface for a base station is shown. The base station provides an alternative mechanism for customizing a medical apparatus or medical device (hereinafter collectively referred to as “medical apparatus”), by allowing the doctor or whoever to set the time between sessions, turn off times (i.e. without using a photosensor), etc. The medical apparatus can also be permitted to be programmed by traditional methods without the use of a base station. The use of a base station can provide an alternative or substitute mechanism for retrieving data from and/or programming the medical apparatus.

The base station can have the ability to allow the operator or person programming or retrieving data to place the medical apparatus in a molded like area for both customizing and retrieving the data stored within the medical apparatus regarding the patient's performance(s) from using the medical apparatus (i.e. measurement readings, etc.). The platform for the base station can be provided with one or more pins that insert in the bottom of a chip and/or electronics of the medical apparatus to allow conductivity to perform these said functions. This feature allows the medical apparatus to communicate with a computer for working with medical data and/or programming, and any adjustments can be made on the computer screen, such as, but not limited to the adjustment capabilities shown on the screen of FIG. 2 or the screen of FIG. 3. Any adjustment(s) made can then be sent to the electronic components of the medical apparatus through the base station. The medical apparatus can be adjusted or transmit its medical data through a physical connection, such as, but not limited to, through a docking or base station of the present invention. It can also receive and/or transmit information (i.e. adjustment, medical data, etc.) through wireless technology.

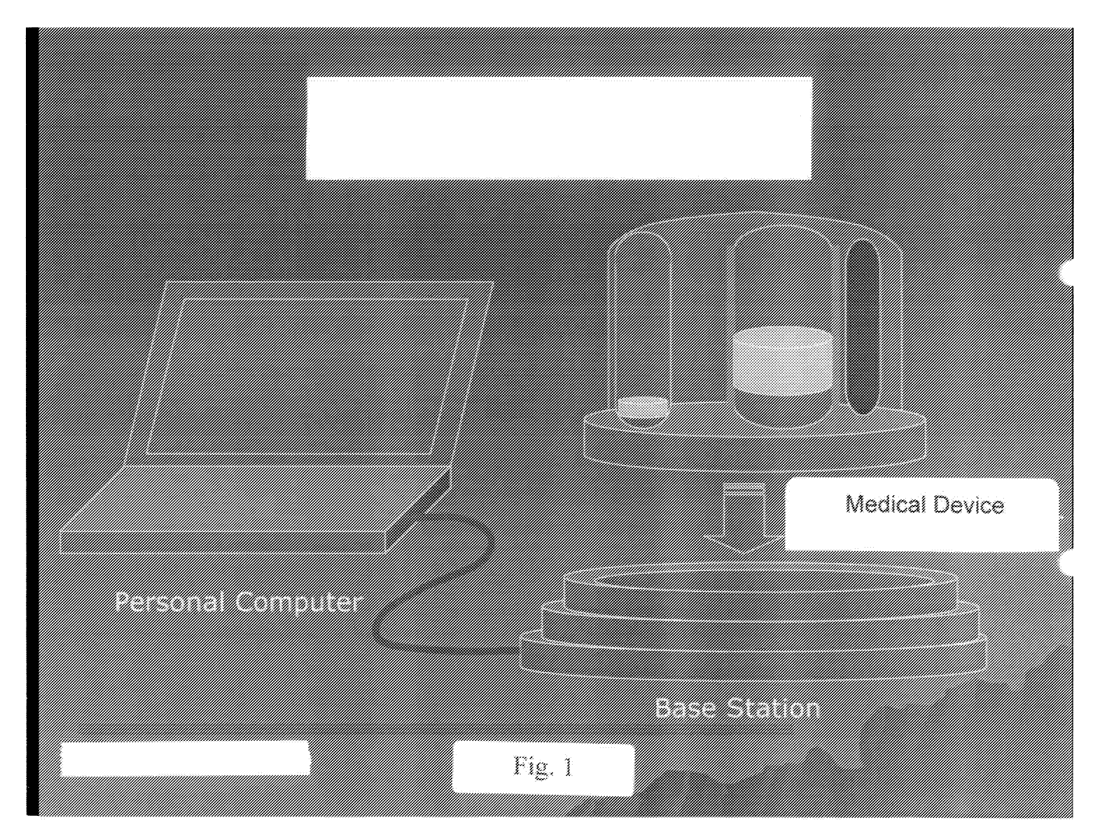

The computer will be able, such as by using a curser controlled by a keyboard, mouse, and/or similar devices, (thus avoiding the need for a large apparatus), to make the programming of the medical apparatus simple. In a preferred embodiment (See FIG. 1) in addition to the computer, a means for holding the medical apparatus (i.e. docking or base station, etc.) is provided to retain the medical apparatus and provide communication thereto, while one uses their mouse and/or keyboard on the computer to adjust or customize the program stored on the medical apparatus or to retrieve data from the medical apparatus.

In one embodiment, the base station can form a mold in the shape of part of the medical apparatus to be connected such as where the programming area is connected. Though not limiting this can be the bottom area of the medical apparatus, often where the electronic components to facilitate the functions of the present invention can be preferably located. The actual shape is not considered limiting. It is preferred that there is some correspondence in shapes between the relevant portions of the medical apparatus and base station, so that the physical mating of the two components is accomplished easier. Alternatively, the base station can be sized and/or shaped to encompass or accommodate more than one size of medical apparatus, and can be manually adjusted to fit or correspond to the size and shape of the apparatus that is connected thereto.

The base station can be a device which facilitates interface to a PC, computer, etc. via a USB cable or other wiring for customization and data download of the medical apparatus, back and forth wireless transmissions can also be communicated through or controlled by the base station. In one embodiment, the medical apparatus can be pressed down onto the base station and the base station can be plugged into the PC, computer, etc. such as through a cable, cord, USB port, etc.

The screen shot illustrated in FIG. 2 shows one non-limiting version of the software interface on the PC or computer which can be used to communicate with a medical apparatus. In this non-limiting example, the following represents a summary of the functionality of the base station/PC(computer)/Software combination. However, it should be recognized that other functions can be provided and are also considered within the scope of the invention.

Time Selector: The upper bar on the screenshot can change the “Active Time” of the unit. Any session times outside of the window will not activate the medical apparatus so that the patient can sleep or otherwise not be disturbed.

Frequency: Standard Frequency of testing can be every 1 Hour, though such is not considered limiting. This can be adjusted in 15 minute intervals or any other desired interval and all are considered within the scope of the invention.

Number of Exercises: The patient can be required to do several exercises (i.e. 4, etc.) exercises every session by default. The number of exercises per session can be adjusted.

Target Volume: This can be an optional parameter. The Hospital or other user can set this target volume to a value appropriate for each patient, if needed, as some patients may be hearing impaired.

Load Configuration: This can be a convenience made possible through the PC screen, which can display the full available options for programming each device. One can configure the screen on the PC for “Standard Patient”, “Senior Male”, “Senior Female”, “Male Child, “Female Child”, etc. or any other category or classification for the individuals or patients, as the screen of the computer can have a picture of an adjustable abilities desired for whatever requirement of programming for any device. There may be some standard set of parameters that the hospital wants to use for different classes according to the patient's medical history. The interface display on the computer screen will or can change automatically, but not limited to, to provide the required designated therapy that can be programmed according to the apparatus being used.

The present invention base station can be used in connection with all types of medical apparatuses. The programmed information from the medical apparatus can be sent to a central computer base in the hospital or other authorized to receive medical or non-medical facility, where a doctor, nurse, or other authorized personnel or individual can retrieve the information where he or she may be located (in the vicinity, in another country, out of state, on a different floor of the hospital, etc.).

Save Configuration: Where values to a group of settings that may be used commonly are set, one can save it off to a file for easy retrieval.

Update Configuration: Write the configuration data to the Serial FLASH, but is not considered limited to FLASH.

Download Data: The medical apparatus stores exercise result data in the Serial FLASH, but is not considered limited to FLASH. This data can be downloaded and saved to a file on the PC, laptop, PDA, computer, etc. by using the Download Data button to confirm patient compliance.

Languages: The base station can be programmed and communicate in various languages and is not limited to any particular language. Information can be viewed in various human languages on the screen and is not considered to any particular one language.

Thus an interface for configuring the medical apparatus via the base station can be provided. The top bar can provide times that the medical apparatus is “Awake”. i.e., it won't prompt the patient at any time outside of this time band so they can sleep. Thus, the medical apparatus can be programmed, customized and adjusted, using the base station, according to the current therapeutic requirements of the patient. In summary, the base station provides a means for programming various types of medical apparatuses and its applicability and benefits are widespread. The base station can comprise a means through a connective source that combines measurements, instructions, specific target goals, predetermined values and intervals for rest periods, that may be needed between particular exercises, in order that those therapeutic guidelines are combined in synthesis with whatever apparatus is being programmed. Depending on the medical apparatus that it is being used in connection with, other features, values, parameters, etc. may be programmed by the base station. This is not only limited to solely therapeutic needs, as a “timer” may be used for various medical requirements, such as, but not limited to, washing a valve, etc. The base station provides a means for programming medical apparatuses with therapeutic guidelines, per the particular requirements needed, for the patient and physician's specifications and provides a way for adjusting or customizing each medical apparatus to the specific needs of the patient.

Thus, the medical apparatus, with or without the base station, can be programmed or customized for any adjustments that relate to the exact therapeutic requirements desired by the one who is setting the medical apparatus, within the guidelines of a qualified therapist or doctor programming each apparatus, including, but not limited to, period of time in which the unit turns off as well as turns back on to allow for a sleep period for the patient.

In a hospital or doctor office setting, a single base station can be provided per floor. The nurse can roll the PC around on a cart with the Base Station and download the performance data. Alternately, the patient's information can be downloaded at whatever area desired by simply forwarding the information to the central communication base station (or other designated base station area) in the hospital itself or any other area designated as the central information retrieval location. Each patient's particular unit can be identified by serial number. Where the medical apparatus is intended to be used for an extended period of time, patient data can be retrieved from the medical apparatus used when the patient goes to his doctor for a follow-up visit.

the following is a non-limiting lists of functionality for the Base Station:

a. Writing Mode
(1) Serial number (correlates to Patient Name); (2) Exercises per set; (3) Sleep time between sets; (4) Target values; (5) Daily active period (lets the patient sleep); and (6) Data is written to a section on the serial flash, but is not considered limited to flash

The Writing Mode can be defined as when the Base Station is used to configure the medical apparatus. All of the above parameters can be programmed into the unit. Writing can be done by clicking on the “Update Data” button in the software, such as, but not limited to, the screen shot shown in FIG. 2.

b. Medical Apparatus Operation
(1) medical apparatus reads serial flash section on wake-up (same chip as stores speech files); and (2) Updates all parameter for program operation can be based on serial flash data, but is not considered limited to serial flash data.

During operation of the medical apparatus, it reads the data written to it by the Base Station. All of the configuration data can be written to the serial Flash (but is not considered limited to Flash) so it is available for the microcontroller to read during operation.

c. Reading Mode

### (1) Serial number; (2) Exercise information (e.g. date, time, exercise number, volume or value achieved, etc.); and (3) Save data to a spreadsheet for record of patient progress/compliance

The Reading Mode can be defined when the patient exercise results are read from the medical apparatus. The results can be stored as a spreadsheet and even plotted out on a chart to see how the patient is doing and to see if they are complying with the instructions of the doctor.

The screen shot illustrated in FIG. 3 shows another non-limiting version of the software interface on the PC or computer which can be used to communicate with a medical apparatus. In this non-limiting example, the following represents a summary of the functionality of the base station/PC(computer)/Software combination.

Load Data button—predefined configurations can be loaded from the hard drive. As a non-limiting example, every male under 30 can have the same Target Volume and number of exercises. Those values may be different than the standard values for a woman over 60.

Save Data button—a current configuration can be stored to a file for later use. As a non-limiting example, a doctor may want all of his patients to have a specific exercise regimen. The fields in the window are filled in and the configuration file can be saved to be associated to the particular doctor as “particulardoctor.usc”.

Update Data button—the data showing in the fields can be loaded into the unit.

Clear Data button—the data on the unit can be erased

Download Data—read all of the recorded exercise results from the unit (e.g. which can be up to 8 weeks worth of data, though such is not considered limiting).

Exit button—quit the program.

Languages—The base station can be programmed and communicate in various languages and is not limited to any particular language. Information can be viewed in various human languages on the screen and is not considered to any particular one language. The base station can also be programmed and communicate in any sound that imitate, resemble, mimic or sound like a human voice, as animals do not speak, but one might programmed the device utilizing an animal speaking for a child.

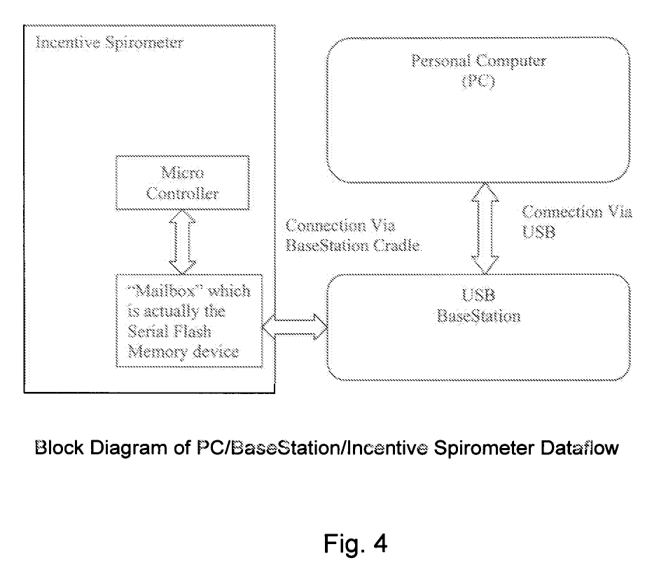

As seen in FIG. 4, another embodiment for the base station is shown and preferably use USB technology for certain connections and is generally designated as base station 400. USB base station 400 can also perform all of the functionality described above and below for the base station illustrated in FIG. 1. Base station 400 provides an interface which facilitates communication personal computer 420 (equipped with a USP port) and a medical device 440, such as, but not limited to an Incentive Spirometer. When used in connection with an Incentive Spirometer, communication via base station 400 serves several functions, which include, but are not limited to: (1) program several default variables such as inspiration volume target, exercise frequency, number of repetitions, “Awake time period” definition, etc. on the Incentive Spirometer; (2) read exercise results that have been stored on the Incentive Spirometer and pass the date, time, and volume of air inspired to the PC where it can be saved to a file or use for another reason, i.e. comparison to previous results, etc.; (3) reprogram the speech files to customize the Incentive Spirometer for different languages or to make the speech files more appropriate for different age groups. These functions can also be performed for other medical apparatuses in addition to an Incentive Spirometer.

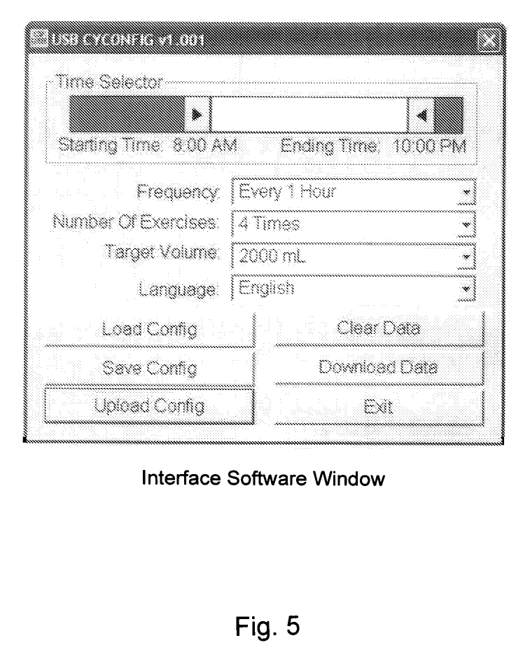

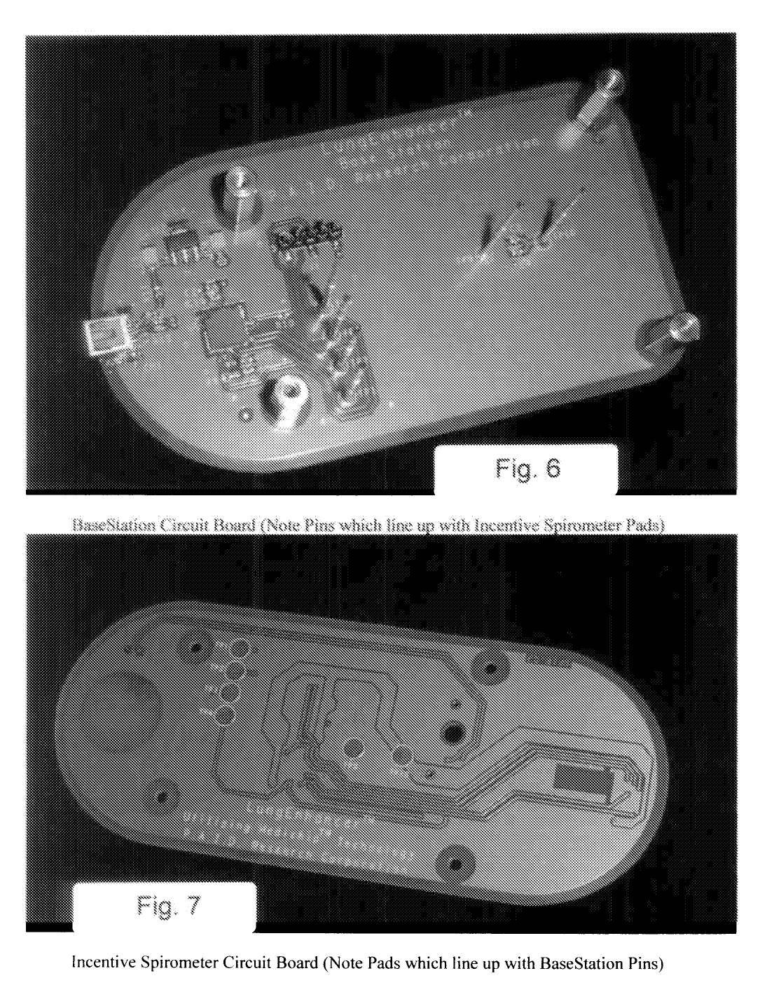

In one embodiment, base station 400 can include a microcontroller, such as, but not limited to a Cypress CY8C24894 USB microcontroller. Other microcontrollers can also be used for one or more of the base stations of the present invention and all are considered within the scope of the invention. The microcontroller serves as a translator between PC 420 and Medical Apparatus 440. In one non-limiting embodiment, the microcontroller serves as a translator between the USB signaling of the PC 420 and the serial peripheral interface (SPI) port of the serial flash device (a memory device) on an Incentive Spirometer. To start the system, the USB port of base station 400 is connected to the USB port of PC 420. Next the interface software on PC 420 is started and the Incentive Spirometer (medical device 440) is connected to base station 400 such as, but not limited to, by placing in the base station cradle. Various commands can then be run by clicking appropriate buttons in an interface software window, such as, but not limited to, software window 500 (FIG. 5) displayed on the screen of PC 420.

Additionally, a microcontroller chip, such as but not limited to the Cypress Chip CY8C24894, is preferably also provided as part of the electronic technology of the medical apparatus, in order to produce a compatible medical apparatus that is capable of being programmed. Thus, the medical device is preferably set up with the chip prior to programming in order for it to be programmed in connection with the base station. Accordingly, the functions of the medical apparatus achieved with its electronic technology, such as, but not limited to, audible, verbal instructions, other sounds, commands, guidance, etc. is preferably previously constructed, stored, loaded, saved, configured, etc. as required for each particular medical apparatus, such that the medical apparatus at the time of being programmed will be in synthesis with the programmer.

Additionally, where pins are provided with the chips, the pins and/or pads which are associated with the medical apparatus chip are preferably set up such that they fit or otherwise correspond to the pins and/or pads of the holding area of the base station or programmer, which can be set up to fit each device accordingly. Accordingly, the medical devices are preferably set up previously and preferably before being connected to the base station such that they are capable of being programmed in accordance with the present invention through the use of the base station.

In another embodiment where the medical device is not compatible or otherwise previously programmed, the base station can be used as a means for loading the necessary software, firm ware or other instructions to made the device capable of being programmed.

Thus, in all embodiments, the base station provides translation such that a computer (i.e. personal computer, etc.) can talk with a medical device or apparatus (which as indicated above is preferably previously programmed such that it is already compatible and able to receive instructions through said base station), such as, but not limited to an Incentive Spirometer. When used with USB technology, the base station translates USB information from the PC into programming/reading commands for the Serial Flash on the medical device (i.e. Incentive Spirometer, etc.). With one non-limiting schematic, a connector on the left (CN1) can be the USB connector which goes to PC 420. The connector wires on the base station board can go to the upper part of the schematic which can be the USB microcontroller, such as, but not limited to the above-mentioned Cypress microcontroller (U1). The microcontroller translates the USB commands and data to Serial Flash commands and date which then proceed to the test points on the right side of the schematic (TP1, 2, 3, 4, 9 and 10). The points listed as test points can be connected to pins (preferably spring-loaded pins) which make contact with the medical device (i.e. incentive spirometer, etc.) when the device is appropriately connected to the base station, such as by being placed in the base station cradle. These pins provide the base station access to the serial flash on the medical device. The PC or other computer can then program, configure, read and otherwise communicate with the medical device (i.e. Incentive Spirometer, etc.).

Thus, some of the non-limiting advantages of a base station, include, but are not limited to the following: (1) can take relatively expensive parts such as a USB connector and USB microcontroller from a “high-runner” medical device (I.e. Incentive Spirometer, etc.) and moves it to a board that can be produced in lower quantities which can allow for a relatively significant reduction in unit cost for the medical device (i.e. Incentive Spirometer, etc.); and (2) impedes tampering by not providing the patient universal I/O port such as the USB connector. As mentioned above, in a preferred (though not limiting) embodiment, all communications can be done via pads on the Incentive Spirometer (medical device) and pins on the base station. If the USB connector were provided on the Incentive Spirometer (medical device), patients could plug the Incentive Spirometer (medical device) into their laptop to see what happens, which could result in damage to the Incentive Spirometer (medical device).

Rather than being physically connected, some or all of the three main components (base station, computer and medical device) can communicate with each through wireless technology such as being Bluetooth enabled, Wi Fi, etc.

Accordingly, with the present invention can be used to program the medical apparatus for volumes, ratios, time segments and individual requirements in relationship to the therapeutic sessions for what the medical apparatus is being used for and/or whatever programmable function suites the apparatus as needed. Furthermore, the present invention also permits for data retrieval from the medical apparatus as well as an average over any given period such as, but not limited to, what was achieved during the week, to permit a doctor to make an accurate prognosis. However, it should be recognized that data can also be retrieved from the medical apparatus by the present invention to simply show what is being done on a daily basis.

It should be recognized the present invention is not considered limited to use with any specific type of medical apparatus or medical device. It will be seen that the objects set forth above, and those made apparent from the foregoing description, are efficiently attained and since certain changes may be made in the above construction without departing from the scope of the invention, it is intended that all matters contained in the foregoing description shall be interpreted as illustrative and not in a limiting sense. The instant invention has been shown and described herein in what is considered to be the most practical and preferred embodiment.

What is claimed is:

1. A system for retrieving information from and programming a medical apparatus, comprising:
a medical apparatus having technology capable of being programmed and obtaining patient information;
a computer independent of said medical apparatus; and
a base station in communication with said medical apparatus and said computer, said base station having technology compatible with the technology of said medical apparatus.

2. The system of claim 1 wherein said medical apparatus is programmed from instructions originated from the computer and received by said medical apparatus through said base station.

3. The system of claim 1 wherein said base station having a cradle-like receiving area for receipt of said medical apparatus.

4. The system of claim 1 wherein said base station is in communication with said medical apparatus through USB technology.

5. The system of claim 1 wherein said base station is in communication with said medical apparatus through wireless technology.

6. The system of claim 5 wherein said wireless technology is Bluetooth technology.

7. The system of claim 5 wherein said wireless technology is WiFi technology.

8. The system of claim 1 wherein said medical apparatus is an Incentive Spirometer.

9. The system of claim 1 wherein said computer is a personal computer.

10. A system for retrieving information from and programming an Incentive Spirometer with a separate personal computer, comprising:
an Incentive Spirometer incorporating technology to permit said Incentive Spirometer to be programmed based on a patient's therapeutic requirements and for obtaining and storing information relating to the patient's performance using the Incentive Spirometer obtaining patient information;
a computer independent of said medical apparatus; and
a base station in communication with said Incentive Spirometer and said computer, said base station having technology compatible with the technology of said Incentive Spirometer.

11. The system of claim 10 wherein said computer is a personal computer.

12. The system of claim 10 wherein said Incentive Spirometer is programmed from instructions originated from the computer and received by said Incentive Spirometer through said base station.
